(19) United States
(12) Patent Application Publication
(10) Pub. No.: US 2009/0281833 A1
(43) Pub. Date: Nov. 12, 2009

(54) SYSTEM AND METHOD FOR SECURE MULTI-PARTY MEDICAL CONFERENCING

(75) Inventor: SHRINIVAS BOPPANA, RICHARDSON, TX (US)

Correspondence Address: HOWISON & ARNOTT, L.L.P, P.O. BOX 741715, DALLAS, TX, 75374-1715, US

(73) Assignee: TELE VIDEO MD, INC., RICHARDSON, TX (US)

(21) Appl. No.: 12/118,142

(22) Filed: May 9, 2008

(51) Int. Cl. A61B 5/00

(52) U.S. Cl. 705/3

(57) ABSTRACT

A system and method for providing secure medical conferencing are provided. In one example, the system includes a database and a medical services authentication server (MSAS) coupled to the database and a packet network. The database includes medical information and the MSAS includes instructions for facilitating the medical conferencing, including enabling medical service consumers to identify and connect directly to medical service providers via the packet network using information from the database.

### TECHNICAL FIELD

The present disclosure relates generally to medical communications, and in particular, to a method for providing secure multi-party medical conferencing.

### BACKGROUND

The Health Insurance Portability and Accountability Act (“HIPAA”) was enacted by the U.S. Congress in 1996. Title II of HIPAA, otherwise known as the Administrative Simplification (“AS”) provisions, requires, inter alia, the establishment of national standards for electronic health care transactions. The AS provisions are designed to encourage the widespread use of electronic data interchange in the US healthcare system while addressing the security and privacy of health data.

Per the requirements of Title II, the Department of Health and Human Services has promulgated five rules regarding Administrative Simplification. Included in the five rules regarding Administrative Simplification are “The Privacy Rule” and “The Security Rule.” Though the rules complement each other, the Privacy Rule pertains to all Protected Health Information (“PHI”) while the Security Rule deals specifically with Electronic Protected Health Information (“EPHI”).

The Privacy Rule, which took effect on Apr. 14, 2003, establishes regulations for the use and disclosure of PHI. PHI is any information about health status, provision of health care, or payment for health care that can be linked to an individual. As such, PHI is interpreted broadly to include any part of a patient's medical record. Any entity subject to the guidelines of HIPAA must disclose PHI to facilitate treatment, payment, or health care operations if the entity has obtained authorization from the individual. However, whenever PHI is disclosed, the entity disclosing the PHI must make reasonable efforts to disclose only that information which is required to achieve the sought purpose.

The Security Rule, which took effect on Apr. 21, 2003, lays out three types of security safeguards required for compliance: administrative, physical, and technical. Administrative safeguards are policies and procedures designed to clearly show how the entity will comply with the act. Such policies and procedures should include, inter alia, a clear identification of employees or class of employees who will have access to EPHI; ensure third party vendors (when applicable) have a framework in place to comply with HIPAA; and an emergency backup plan for data with disaster recovery procedures.

Many entities comply with HIPAA by not transmitting electronic files containing any EPHI or by transmitting physical files with PHI. Additionally, these entities require the individual to sign forms to release the PHI to third parties. These procedures can be cumbersome in emergency conditions.

Additionally, securing a medical conference with a Medical Service Provider (“MSP”) (e.g., a doctor) in a situation that is not an emergency can be quite cumbersome as well. For example, providing medical care to a child at school may involve multiple parties (including an MSP) even if the problem is not serious.

Accordingly, what is needed are systems and methods that can be deployed to allow a multi-party medical conference in which two or more parties in separate locations can discuss medical treatments for a patient in a secure environment.

### SUMMARY

In one embodiment, the present disclosure provides a system for providing medical conferencing for a plurality of parties by a medical conferencing services provider, the plurality of parties including a medical service provider (MSP) and a consumer of medical services. The system comprises a database that includes an electronic storage medium having a plurality of records stored thereon. The plurality of records includes a first profile, a first identifier, and a first password corresponding to the MSP, a second profile, a second identifier, and a second password corresponding to the consumer, and an MSP list containing a plurality of MSP identifiers corresponding to a plurality of MSPs on the MSP list, wherein one of the plurality of MSP identifiers is the second identifier and wherein the MSP list includes at least one MSP identifier not added to the MSP list by the consumer. The system also includes a medical services authentication server (MSAS) coupled to the database and a packet network. The MSAS includes an electronic storage medium having a plurality of instructions stored thereon for execution by the MSAS. The plurality of instructions includes instructions for receiving the first password from a first device via the packet network and authenticating the first device as authorized to access the medical conferencing services provider based on the first password; receiving the second password from a second device via the packet network and authenticating the second device as authorized to access the medical conferencing services provider based on the second password; sending the MSP list and corresponding status information to the second device via the packet network after authenticating the second device, wherein the corresponding status information includes a status of the first device corresponding to the first identifier as available for contact by the second device; and sending routing information to the second device via the packet network after authenticating the second device, wherein the routing information includes network address information needed by the second device to establish a secure, point-to-point communication session directly with the first device.

### BRIEF DESCRIPTION OF THE DRAWINGS

For a more complete understanding, reference is now made to the following description taken in conjunction with the accompanying drawings in which:

FIGS. 1 and 2 are block diagrams illustrating exemplary medical communications involving three parties;

FIG. 3 is a block diagram illustrating one embodiment of a secure multi-party medical conferencing session;

FIG. 4a is a sequence diagram illustrating one embodiment of a method for establishing a secure multi-party medical conferencing session;

FIG. 4b is a flow chart illustrating one embodiment of a method for establishing a secure multi-party medical conferencing session;

FIG. 5 is a block diagram illustrating one embodiment of a system that may support secure multi-party medical conferencing;

FIG. 6a is a block diagram illustrating a more detailed embodiment of a portion of the system of FIG. 5;

FIG. 6b is a block diagram of one embodiment of a device that may be used within the system of FIG. 5;

FIG. 7 is a sequence diagram illustrating one embodiment of a method in which medical records may be transferred during a secure multi-party medical conferencing session;

FIG. 8 is a flow chart illustrating one embodiment of a method for using an auto-attendant in a secure multi-party medical conferencing system;

FIG. 9 is a flow chart illustrating one embodiment of a method for establishing a secure multi-party medical conferencing session; and

FIG. 10 is a flow chart illustrating one embodiment of a method for establishing a secure multi-party medical conferencing session involving a specialist.

### DETAILED DESCRIPTION

It is understood that the following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Furthermore, the figures are not necessarily drawn to scale, and in some instances the drawings have been exaggerated and/or simplified in places for illustrative purposes only.

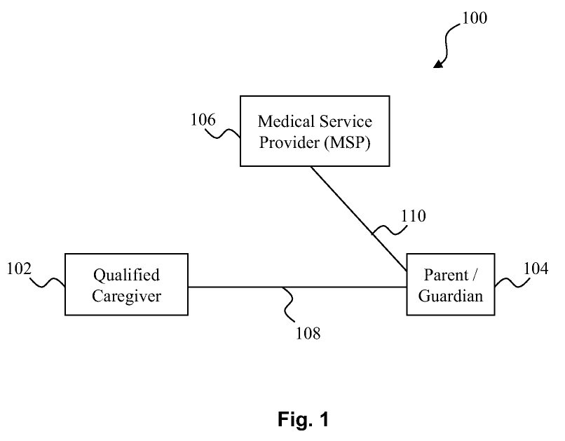

Referring to FIG. 1, one embodiment of a medical communication scenario 100 is illustrated that involves three parties. In the present example, a child (not shown) starts to feel ill while at school. Alternatively, the child could have been injured while at school. The child is taken to a school nurse, who is a medically qualified caregiver 102. The school nurse 102 determines that the child should be seen by an MSP (e.g., a physician) 106. The school nurse 102 contacts the child's parent or legal guardian (hereinafter “parent”) 104 via a communication channel 108. For example, the communication channel 108 is typically a telephone communication network. The school nurse 102 informs the parent 104 of the nature of the child's illness or injury. Then, if needed, the school nurse 102 informs the parent 104 that the child should be seen by an MSP.

Thereafter, the parent 104 initiates contact via a communication channel 110 (e.g., a telephone communication network) with their MSP 106. In the present example, the MSP is a primary care physician (“PCP”). The parent 104 schedules a time when the MSP 106 is available to see the child and the parent is available to take the child to the MSP's location (or when the parent can make arrangements for someone else to take the child). Frequently, the parent 104 must then leave work or whatever activity in which the parent 104 was engaged, go to the school, and take the child home or to the MSP's location. As often happens, if the MSP 106 is not able to see the child on the same day on which the parent 104 called, the parent 104 must take additional time away from work or other planned activities to take the child to the MSP at the date and time scheduled.

This can be a particularly time consuming process if the child suffers from chronic illness and may be particularly frustrating if the MSP 106 merely requires some basic medical information (e.g., temperature and blood pressure) in order to make an accurate diagnosis. Additionally, the child's illness or injury may be such that the MSP 106 merely needed to prescribe some medication that the parent 104 could have picked up on the way to get the child from the school. However, such solutions are not generally available because the school nurse 102 is usually not authorized to administer medical care without the approval of the parent 104 and is also generally not permitted to diagnose injuries and illnesses.

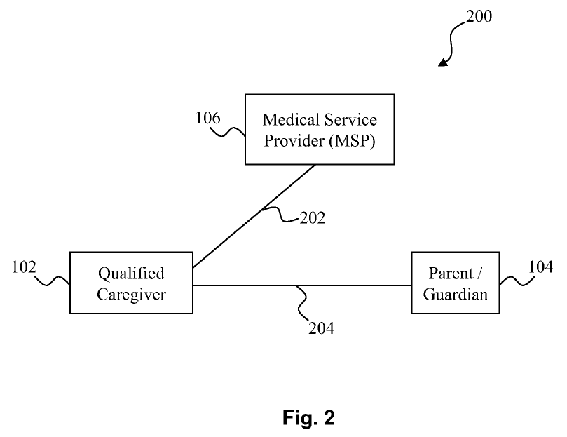

Referring now to FIG. 2, an embodiment of another medical communication scenario 200 involving three parties is illustrated. In the present example, when the ill or injured child reports to the qualified caregiver 102 (e.g., the school nurse), the school nurse initiates contact with an MSP 106 via communication channel 202. The MSP 106 may be the child's PCP or may be another physician (e.g., a physician on contract with the school). The communication channel 202 may be established via a dedicated line (e.g., a T1 connection, ISDN connection, or telephone communication network) between the MSP 106 and the school nurse 102.

In this example, the school nurse 102 is permitted to communicate with the MSP 106 regarding the injuries or symptoms of the child. The MSP 106 can then instruct the school nurse 102 regarding the performing of tests or the obtaining of medical readings required to make an accurate diagnosis. Accordingly, the MSP 106 is able to make a diagnosis and determination as to whether the child can be treated at the school or whether the child should be taken to a medical facility, such as a doctor's office or emergency room. However, the nurse 102 and/or the MSP 106 may still be required to communicate with the parent 104 to inform the parent of the illness or injury of the child and to obtain authorization from the parent to treat the child. Such communications may be accomplished via a communication channel 204 (e.g., a telephone communication network).

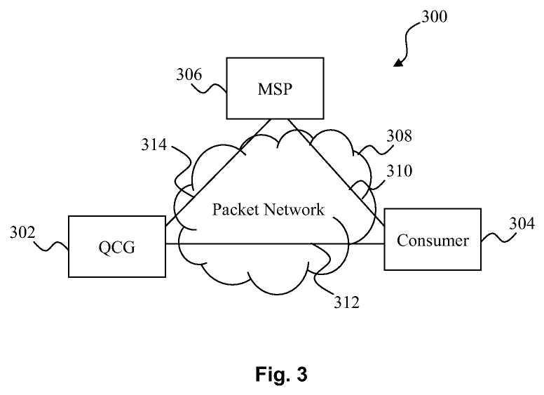

Referring to FIG. 3, one embodiment of a secure multi-party medical conferencing system 300 is illustrated. In various embodiments, the multi-party medical conferencing system 300 enables multiple parties, such as a qualified caregiver 302, a medical services consumer 304, and one or more MSPs 306 to be connected via a packet network 308, such as a Global Communication Network (“GCN”) like the Internet. In such embodiments, the multi-party medical conferencing system 300 may establish secure voice, video, and data communications via a hybrid peer-to-peer system. One example of such a hybrid peer-to-peer system is developed by Damaka, Inc. (located in Richardson, Tex.), and is disclosed in U.S. patent application Ser. No. 11/081,068, which is hereby incorporated by reference in its entirety. It is understood that the hybrid peer-to-peer system developed by Damaka is used herein for purposes of example only, and that other systems may be used to implement some or all of the present disclosure.

In the present example, the consumer 304 may subscribe to a multi-party medical conferencing service offered by a medical conferencing services provider. The consumer 304 may join such a service by entering registration information and paying an initial fee. The initial fee may be for registration and other fees, such as a periodic fee for continued monthly subscription, may also be required. A service term contract may be required or services may be rendered on an “as needed” basis. It is understood that these are merely examples and that many different types of access may be provided to the multi-party medical conferencing service.

When the consumer 304 registers with the multi-party medical conferencing service, the consumer may provide information necessary to complete a member profile. The member profile may contain demographic information such as, but not limited to, the consumer's age, address, contact information, marital status, and payment information (e.g., financial institution information to be utilized for payments such as automatic withdrawal). The consumer 304 may also provide certain health related information including, but not limited to, the names of the consumer's primary care physician and insurance provider. The consumer 304 may also provide information for any dependents of the consumer, such as a spouse, children, or legal wards. The information relating to the dependents of the consumer 304 may be saved in the consumer's profile or additional profiles may be created for each respective dependent of the consumer. The consumer 304 may register with the multi-party medical conferencing service by calling a specified telephone number, submitting a completed paper form by mail, facsimile, or email, or by accessing a registration server and completing an online registration process.

Upon registration, the consumer 304 may be provided with authentication credentials, such as a user identifier (ID) and password. In some embodiments, the consumer 304 may supply a proposed user ID and/or password. In such embodiments, the multi-party medical conferencing service may then confirm that the user ID and/or password are unique and, if not unique, may prompt the consumer 304 to provide another user ID/password until a unique user ID/password is assigned to the consumer. Additionally, the consumer 304 may be provided with an access code, which may be unique. This access code can be different than the user ID and password, or the access code and the user ID can be related. In some embodiments, the consumer 304 provides a proposed access code. In such embodiments, the multi-party medical conferencing service may confirm that the access code is unique and, if not unique, may prompt the consumer 304 to provide another access code until a unique access code is assigned to the consumer.

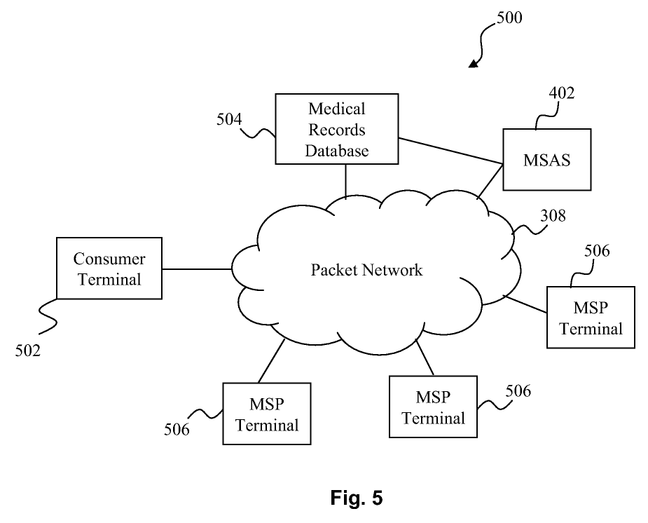

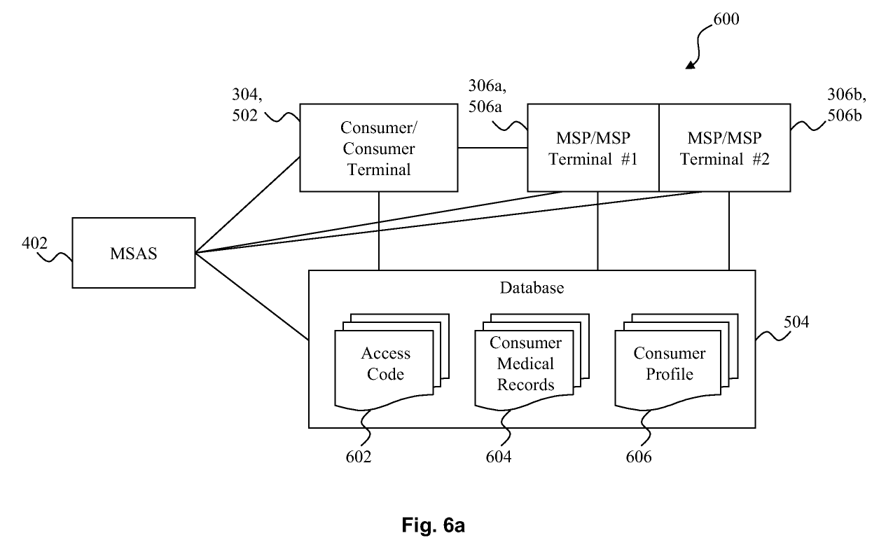

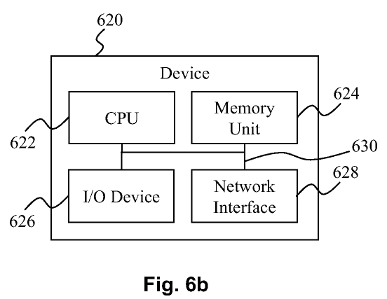

The user ID, password, and access code are stored in a database (discussed below with respect to FIGS. 5 and 6) with the member profile that is created using the information provided by the consumer 304 during registration. The member profile is saved in the database with a relationship corresponding to the user ID, password, and/or access code. If profiles were created for the consumer's dependents, the dependents' profiles are also saved in the database with a relationship corresponding to the consumer's profile. Furthermore, in some embodiments, the consumer 304 is provided with additional access codes that uniquely correspond to the dependent profiles.

After the consumer 304 has successfully registered, the consumer is provided with client software to be installed on a device (not shown) that is capable of accessing the multi-party medical conferencing service offered by the medical conferencing services provider via the packet network 308. The device (i.e., an “endpoint” in terms of previously incorporated U.S. patent application Ser. No. 11/081,068) can be a personal computer, personal data assistant (“PDA”), cellular phone, or any other device that is capable of accessing the packet network 308. The client software is designed to enable the device of the consumer 304 to connect to the multi-party medical conferencing service as a patient terminal such that the consumer, when seeking medical services, is able to view information (e.g., a list of MSPs) provided by the medical conferencing services provider.

Additionally, some embodiments provide for the MSP 306 to subscribe to the medical conferencing services provider. For example, the MSP 306 can be a physician, surgeon, or other doctor authorized to practice medicine. The MSP 306 may be required to pay a fee upon registering for the service. Alternatively, the MSP 306 may receive compensation for agreeing to register with the service depending upon the laws within a particular state or country.

When the MSP 306 seeks to register with the service, the MSP provides information necessary to complete an MSP profile. The information provided by the MSP 306 may include, but is not limited to, demographic information such as address and types of medical services offered (i.e., types of medicine practiced such as general family medicine, pediatrician, and surgery). The types of medical services offered (i.e., the medical specialty or specialties of the MSP 306) may be stored in the MSP profile with a unique specialty code corresponding to each specialty offered. In some embodiments, the information provided by the MSP 306 includes insurance plans accepted, promotional offers currently available from the MSP, and/or insurance plans offered by the MSP. In some embodiments, when the MSP 306 seeks to register with the medical conferencing services provider, the MSP is first required to be certified by a third party screening service. The third party screening service may provide services that include, but are not limited to, verifying the credentials of the MSP 306, maintaining a copy of licenses corresponding to the MSP, confirming legal requirements for medical practice of the state/country where the MSP is located, obtaining any records of disciplinary action against the MSP, and any positive or negative patient feedback regarding the MSP.

Upon registration, the MSP 306 (who may be referred to herein as a “member MSP”) may be provided with an MSP ID and password. In some embodiments, the MSP 306 supplies a proposed MSP ID and password. In such embodiments, the multi-party medical conferencing service may confirm that the MSP ID is unique and, if not unique, may prompt the MSP to provide another MSP ID until a unique MSP ID is assigned to the MSP. The MSP ID is uniquely associated with the MSP profile.

After the MSP 306 has successfully registered, the MSP 306 is provided MSP client software to be installed on a device that is capable of accessing the Internet 308 (e.g., a device such as the device described above with respect to the consumer). The MSP client software enables the device of the MSP 306 to connect to the medical conferencing services provider as an MSP terminal. Accordingly, when the MSP 306 seeks to sign-on to the medical conferencing services provider, the MSP provides the MSP ID and password. Thereafter, the MSP profile is accessed and the MSP 306 is signed-on to the medical conferencing services provider as an MSP.

In some embodiments, the MSP 306 may register with a third party MSP agency. In such embodiments, the third party MSP agency may provide a list of MSPs to the medical conferencing services provider for medical consultations. For example, the third party MSP agency may establish contracts or agreements with multiple MSPs 306 to provide medical consultation via the medical conferencing services provider. During the registration process, the MSP 306 provides the third party MSP agency with information necessary to complete the MSP's profile. The third party MSP agency pre-screens the MSP 306 by reviewing MSP qualification information such as, but not limited to, credentials, experience, work history, malpractice insurance, malpractice claims, references, and patient comments. The third party MSP agency verifies the credentials and, when verified, provides the MSP's name and profile information to the medical conferencing services provider. Accordingly, the third party MSP agency is able to provide assurance to the medical conferencing services provider that the registered MSP 306 (and other MSPs) possess the proper credentials.

In some embodiments, the third party MSP agency provides to the medical conferencing services provider a database list, with credentials, of the registered MSPs 306 that are available for consultation. In additional embodiments, the MSPs 306 are provided to the medical conferencing services provider with a schedule of availability indicating particular dates and times that respective MSPs 306 are available for consultation.

In some embodiments, the medical conferencing services provider provides the MSP 306 with the MSP client software for use on the MSP device. In other embodiments, the medical conferencing services provider provides the third party MSP agency with the MSP client software and the third party MSP agency provides the software to the MSP 306. The third party MSP agency may provide the MSP 306 with the MSP's ID and password, or the MSP ID and password can be provided by the medical conferencing services provider or the MSP as previously described.

Further embodiments of the secure multi-party medical conferencing provide for medically qualified caregivers (QCGs) 302 to subscribe to the medical conferencing services provider. The QCG 302 may represent many different entities that are trained and certified to provide limited medical care such as, for example, a nurse, a nurse practitioner (“NP”), a registered nurse (“RN”), and medical assistant (“MA”). The QCG 302 may also represent the above entities in various settings such as retail, business, school, company, nursing home, hospital, medical kiosk or medical station, medical treatment center, or home health care agency. The QCG 302 may be required to pay a fee upon registering for the service. When the QCG 302 seeks to register with the service, the QCG 302 provides information necessary to complete a QCG profile. The information provided by the QCG 302 includes, but is not limited to, demographic information such as name, address, and type of qualified caregiver (e.g., a school with a nurse).

When the QCG 302 seeks to register with the medical conferencing services provider, the QCG may be required to obtain certification from a third party screening service. The third party screening service can provide services that include, but are not limited to, verifying the QCG 302 credentials, maintaining a copy of licenses for the QCG, and confirming the QCG's legal eligibility to operate as a QCG as part of the medical conferencing services provider in the state/country where the QCG is located.

Upon registration, the QCG 302 may be provided a QCG ID and password. In some embodiments, the QCG 302 may supply a proposed QCG ID and password. In such embodiments, the medical conferencing services provider may confirm that the QCG ID is unique and, if not unique, may prompt the QCG 302 to provide another QCG ID until a unique QCG ID is assigned to the QCG. The QCG ID may be uniquely associated to the QCG profile.

After the QCG 302 has successfully registered, the QCG is provided with QCG client software to be installed on device that is capable of accessing the Internet 308 (e.g., a device such as the device described above with respect to the consumer 304). The QCG client software is configured to cause the device of the QCG 302 to connect to the medical conferencing services provider as a QCG terminal. When the QCG 302 seeks to sign-on to the medical conferencing services provider using the device, the QCG provides the QCG ID and password. Thereafter, the QCG profile is accessed and the QCG 302 is signed-on to the medical conferencing services provider as a QCG. Although separate client software is described for each of the consumer 304, MSP 306, and QCG 302, it is understand that identical software may be provided to each one and the medical conferencing services provider may distinguish between the consumer, MSP, and QCG based on login information.

In some embodiments, the QCG 302 may represent a franchise of medical offices, medical stations, or medical kiosks. The franchise of medical offices, medical stations, or medical kiosks (hereinafter “the QCG franchise”) may be owned by one or more MSPs 306 or a third party agency. For example, the QCG franchise may be operated by attendants and at least one individual who is a QCG as defined previously. At least one QCG may be present at all times during the hours of operation of the QCG franchise.

The QCG franchise may be designed to serve consumers who are subscribers of the medical conferencing services provider as well as individuals who are not subscribers of the medical conferencing services provider. Individuals who are subscribers of the medical conferencing services provider may enter one of the medical offices, medical stations, or medical kiosks and provide their user ID to obtain access to the medical conferencing services provider. Individuals who are not subscribers of the medical conferencing services provider may enter one of the medical offices, medical stations, or medical kiosks and, upon payment of a fee, be given access to the medical conferencing services provider.

Further, the QCG franchise may include patient terminals (e.g., devices as described previously that consumers and other individuals may use to access the medical conferencing services provider to conduct a secure medical conference). The devices may be contained in private rooms such that, if needed, a QCG 302 (e.g., a nurse or medical technician) can be present in the room with the consumer 304 and provide physical services (e.g., taking temperature or blood pressure readings) requested by the MSP 306. The QCG franchise may further include QCG terminals where one or more QCGs 302 who staff the QCG franchise location can provide qualified caregiver medical services to a “walk-in” patient as part of a secure multi-party medical conferencing session with an MSP 306.

With continued reference to FIG. 3, one embodiment of a communication scenario that may occur using the secure multi-party medical conferencing system 300 is now described. A child (not shown) reports to the QCG 302 (e.g., a school nurse) regarding an illness or injury. The QCG 302 contacts the consumer 304 (e.g., the child's parent) and informs the consumer that the child is sick or injured. For example, the QCG 302 may place a telephone call to the consumer 304 or, in some embodiments, may log in to the medical conferencing services provider and notify the consumer 304 of the child's injury/illness via the medical conferencing services provider.

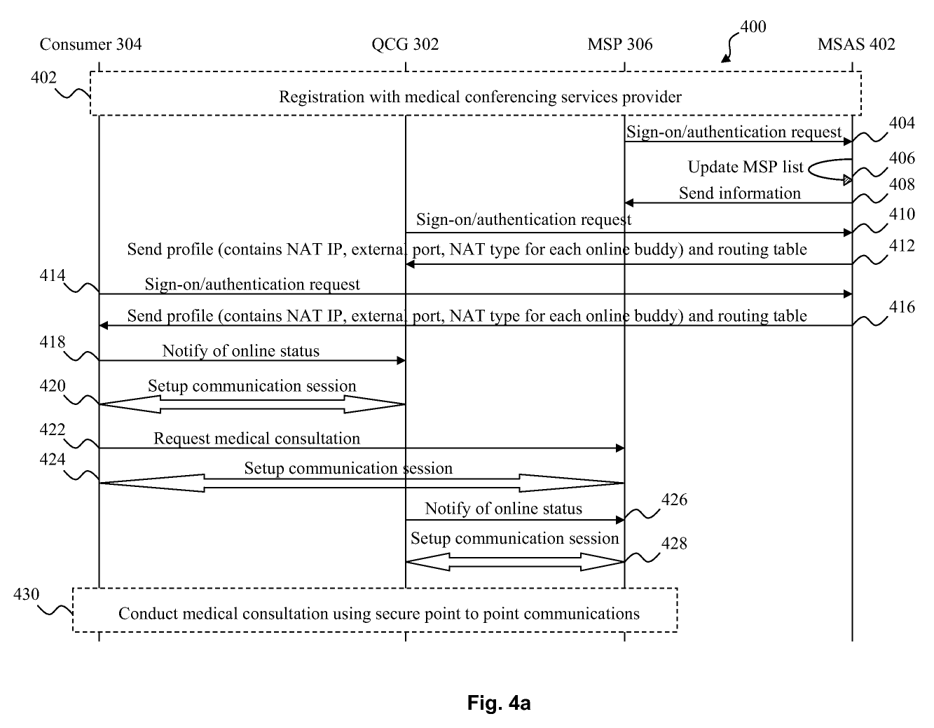

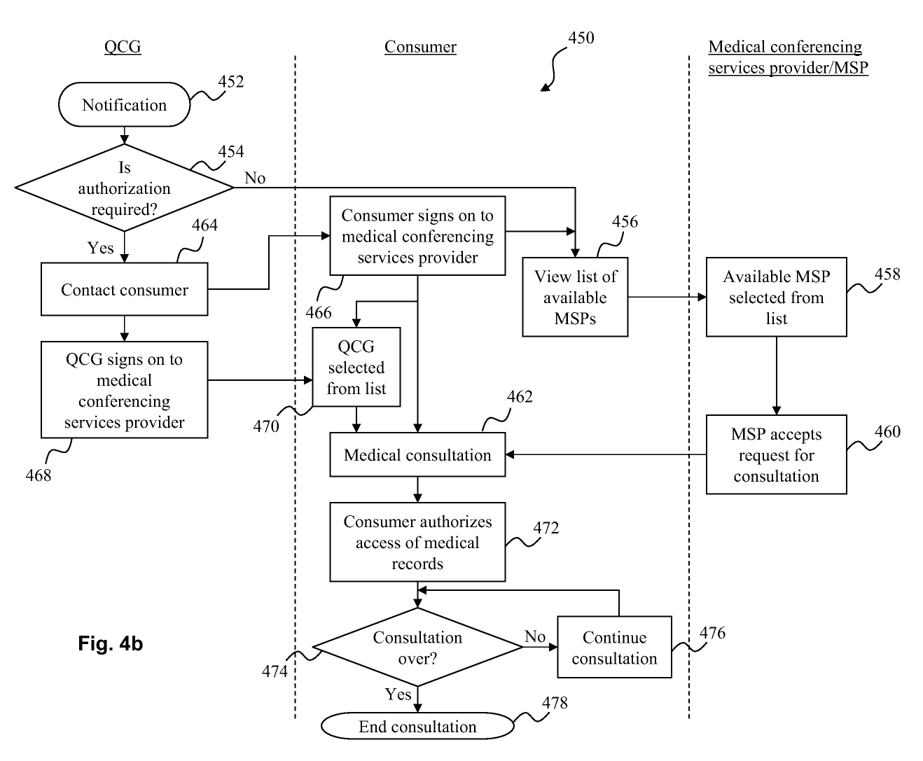

The consumer 304 accesses the medical conferencing services provider to establish a session for secure multi-party medical conferencing via the packet network 308. Upon receiving the consumer's log in credentials, the medical conferencing services provider provides the consumer 304 with a list of available MSPs. In the present embodiment, the medical conferencing services provider may generate the list of available MSPs from the member MSPs who are currently signed-on to the medical conferencing services provider. For example, whenever an MSP 306 signs-on to the medical conferencing services provider, the medical conferencing services provider may update the list of available MSPs to show that the MSP is available for medical consultation. Additionally, the medical conferencing services provider may access the specialty code in the MSP profile corresponding to the MSP ID of the MSP 306 and may include the specialty in the list. The list generated by the medical conferencing services provider is saved in the database and can be provided to the consumer 304 in a number of different formats, as will be discussed herein below with respect to FIGS. 4a, 4b, 5 and 6.

It is understood that the list of MSPs viewed by the consumer 304 may not include all possible MSPs, but instead may represent a filtered view of available MSPs. For example, the medical conferencing services provider may present to the consumer 304 only those MSPs in the consumer's geographic area or may use other criteria to filter the list. Additionally or alternatively, the consumer 304 may select filtering criteria to select only a portion of all available MSPs for viewing.

When the consumer 304 views the list provided by the medical conferencing services provider, the consumer can review the list of MSPs to determine which MSPs that are available for consultation can provide the medical services required. For example, the consumer 304 may choose to look for a family practitioner and so may filter or otherwise review the list for MSPs corresponding to a family practitioner specialty type. When the consumer 304 decides upon a particular MSP 306, the consumer selects the MSP. (A more specific example describing details of accessing the medical conferencing services provider and selecting one or more MSPs will be discussed below with respect to FIG. 4a). Thereafter, a secure communication channel 310 is established between the consumer 304 and the MSP 306 via the packet network 308 using their respective devices containing the software provided by the medical conferencing services provider. It is understood that the communication channel 310 may include voice, video, data, and other types of information. In some embodiments, communications across the secure communication channel 310 may be encrypted. In other embodiments, the MSP 306 may be required to accept a connection request from the consumer 304 prior to the establishment of the secure communication channel 310.

In the present example, the school is registered with the medical conferencing services provider as a QCG 302. Accordingly, the consumer 304 is able to establish a communication channel 312 with the QCG 302 (e.g., the school nurse) (the details of connecting to the school will be discussed in detail with respect to FIG. 4a). Alternatively, as previously described, the QCG 302 may initiate contact with the consumer 304 via the medical conferencing services provider. It is understood that the QCG 302 may remain signed-on to the medical conferencing services provider throughout the day or may access the medical conferencing services provider at specific times or when a medical consultation is needed. After the consumer 304 establishes contact with the QCG 302 (assuming that contact was not already established), the QCG may be included in communications occurring over the communication channel 310. As will be described below, this may involve the establishment of a third communication channel 314. It is understood that the described connection sequence is exemplary and that the QCG 302, consumer 304, and MSP 306 may be connected in any order or simultaneously.

After the secure connections 310, 312, and 314 are established, the consumer 304 can authorize the QCG 302 and MSP 306 to provide medical services to the child. Furthermore, the medical conferencing services provider (either itself or through the client devices of the QCG 302, consumer 304, and MSP 306) is able to maintain a record of the parent's authorization for medical services by recording text, video, voice, etc. Additionally, the MSP 306 can question the QCG 302 regarding the symptoms the child is experiencing or the extent of the child's injuries. The child may also participate in the session through, for example, voice or video. The MSP 306 is able to direct the QCG 302 to run specific tests or take specific measurements of the child. Additionally, in some embodiments, the medical records of the child can be securely stored and made accessible via the medical conferencing services provider. The MSP 306 can be provided access to the child's medical records via the medical conferencing services provider for purposes of aiding in the diagnosis and treatment of the child. Thereafter, the MSP 306 can make a medical determination regarding the appropriate treatment of the child. The MSP 306 may recommend that the consumer 304 take the child to the child's primary care physician (which may be the MSP 306), to a hospital, or to a specialist. The MSP 306 may also prescribe medication or may advise the consumer 304 of a recommended course of treatment that the consumer 304 may follow at home.

In some embodiments, the consumer 304 is able to connect to a nearby pharmacy via the medical conferencing services provider, which may be registered with the medical conferencing services provider as a pharmacy terminal (not specifically illustrated). In such an embodiment, a pharmacy, franchise of pharmacies, or store with a pharmacy located therein (hereinafter all referred to as a “pharmacy”) may register with the medical conferencing services provider as a pharmacy terminal. The medical conferencing services provider provides the pharmacy with software to connect a device at the pharmacy (e.g., similar or identical to the device described previously with respect to the consumer 304) to the medical conferencing services provider as a pharmacy terminal.

The pharmacy terminal may be configured such that it can be included in the secure communications of the consumer 304 and/or MSP 306. The MSP 306 is then able to prescribe medications to the child and send the prescription directly to the pharmacy via the medical conferencing services provider. This process also allows the MSP 306 and pharmacy to ensure that they understand one another regarding the prescription and allows them to correct any problems. The consumer 304 may pay the pharmacy via the medical conferencing services provider (e.g., using a credit card or financial information stored by the medical conferencing services provider), and may have the medications scheduled for pick-up or delivered to the school, to the consumer's work location, or to the consumer's home. Thereafter, the secure communication channels 310, 312, and 314 may be terminated. Accordingly, the QCG 302 and/or consumer 304 may proceed with the recommended course of treatment, thereby ensuring that the child receives prompt medical care and possibly reducing the amount of time required by the consumer 304 to ensure that such care is received.

Referring now to FIG. 4a, a sequence diagram illustrates one embodiment of a method 400 that may be used to connect multiple parties to a medical conferencing services provider. For purposes of illustration, the QCG 302, consumer 304, and MSP 306 of FIG. 3 are used, but it is understood that fewer or more entities, as well as different entities, may be involved in the method 400. A medical services authentication server (MSAS) 402 is used to provide communications services by the medical conferencing services provider as described below. The MSAS 402 may include one or more processors coupled to a storage medium (not shown). The processor is responsive to medical conferencing services provider server software stored on the storage medium. The server software contains a plurality of instructions executable by the processor that enable the MSAS 402 to aid in establishing secure communications between devices, such as patient terminals, MSP terminals, and QCG terminals. It is understood that the MSAS 402 may represent multiple servers and may be distributed.

As indicated by step 402, in the present example, the QCG 302, consumer 304, and MSP 306 have registered with the medical conferencing services provider as described previously. Accordingly, when the method 400 begins, each of the QCG 302, consumer 304, and MSP 306 have a device configured to execute software capable of connecting to the medical conferencing services provider, and each has established a profile and is authorized to provide and/or consume services offered via the medical conferencing services provider.

In step 404, the MSP 306 authenticates with the medical conferencing services provider by accessing the medical conferencing services provider via the packet network 308. The authentication process may include the MSP 306 providing the MSP ID and password to the MSAS 402.

The MSP 306 may log in to the MSAS 402 at an allotted time or may log in due to an opening (e.g., if a scheduled patient cancels or fails to show up for an appointment). For example, upon logging in to the MSAS 402, the MSP 306 may indicate that he or she will be available for thirty minutes or for some other amount of time. Alternatively, the MSP 306 may simply log in to the MSAS 402 without specifying a particular amount of time, or may log in for a period of time corresponding to regular office hours for the MSP. Accordingly, availability of the MSP 306 may be variable and may be random or may include specified times during the day, on specified dates, or a combination thereof.

Although not shown in FIG. 4a, it is understood that the MSAS 402 may prompt the MSP 306 to provide a “time available” in order to include the duration that the MSP 306 will be available for consulting via the medical conferencing services provider. The MSP 306 may be required to respond with a “time available” period or may by-pass the prompt without defining such a time period. In some embodiments, the MSP 306 can optionally provide the “time available” to the MSAS 402 at sign-on.

In step 406, the MSAS 402 updates the list of available MSPs with the information of the MSP 306. In step 408, the MSAS 402 may respond to the MSP 306 with such information as profiles and one or more routing tables. The profiles may correspond to patients that have requested a consultation or are currently waiting online for a consultation, available pharmacies, and other relevant information. In the present example, the profiles belong only to entities (e.g., consumers, QCGs, and MSPs) registered with the medical conferencing services provider. Accordingly, each registered entity may have the potential of being a “buddy” in terms of previously incorporated U.S. patent application Ser. No. 11/081,068. As described in U.S. patent application Ser. No. 11/081,068, a buddy may be directly contacted by another buddy, but non-buddies (even if registered with the medical conferencing services provider) may not be directly contacted without taking additional steps to add them as buddies. Accordingly, controlling who is listed as a buddy may be used to control access to other entities for a particular QCG 302, consumer 304, and MSP 306. However, not all registered entities may be a buddy and the list of buddies may be filtered based on criteria such as geographic location, participation in a particular insurance plan, etc. Furthermore, the registered entities may be displayed in a hierarchical format that provides a user with a “drill down” menu for purposes of convenience.

The routing table may identify such information as Internet Protocol (IP) address, external port number, and Network Address Translation (“NAT”) type needed to establish communication directly with another registered entity. Although not described herein, it is understood that STUN requests and similar techniques may be used to obtain information needed for the profile and/or routing table, and that some such information may be provided by the MSP terminal to the MSAS 402 during log in.

In steps 410 and 412, the QCG 302 authenticates with the MSAS 402 using the corresponding QCG ID and password. After verifying the QCG ID and password, the MSAS 402 authenticates the QCG 302 as a valid member of the medical conferencing services provider and sends profile and routing table information to the QCG terminal. Settings in the QCG profile or in the QCG client software itself may be used by the QCG client software to determine how the available provider list is displayed to the QCG 302. For example, the QCG 302 may be authorized to access only specified MSPs, and this information may be used to tailor the profile that is sent to the QCG 302 to ensure that the QCG profile contains only information pertaining to the specified MSPs. Although not shown, in some embodiments, the client software of the QCG 302 may be configured to notify the MSP 306 that the QCG is online. Due to the point-to-point nature of the system 300 in the present example, the QCG 302 sends the notification message directly to the MSP 306 without sending it through the MSAS 402.

In step 414 (e.g., after being contacted by the QCG 302 that the consumer's child needs medical care), the consumer 304 authenticates with the medical conferencing services provider via the packet network 308. The consumer 304 may send a sign-on/authentication request to the MSAS 402 that includes the consumer's ID and password. In response and after verifying the consumer's authentication information, the MSAS 402 authenticates the consumer 304 as a valid member of the medical conferencing services provider and sends profile and routing table information to the consumer in step 416.

The profile information contains information relating to entities registered with the medical conferencing services provider. It is understood that a list of “buddies” sent to the consumer 304 may be filtered based on criteria such as geographic location, participation in a particular insurance plan, etc. In addition, the consumer 304 may have added entities of particular relevance (e.g., the QCG 302 who is the school nurse at the child's school). As described above, the registered entities may be displayed in a hierarchical format that provides a user with a “drill down” menu for purposes of convenience. The routing table may identify such information as IP address, external port number, and NAT type needed to contact the buddies of the consumer 304.

In some embodiments, the MSAS 402 may send the available provider list (i.e., an MSP list) stored in the database to the consumer 304 as part of the profile or separately. The consumer 304, using the client software or other means, may configure how the available provider list is displayed and may view the available provider list sorted by any number of data fields including, but not limited to, name, field, location, or specialty (based upon specialty code). The available provider list may include a listing of all the MSPs 306 that are currently signed-on to the medical conferencing services provider and are currently available for consultation. The list may include a time available indicator for providing a time period during which each MSP 306 will be available. The list of available MSPs may be updated periodically by the MSAS 402 or may be updated locally on the consumer's device as buddy MSPs log onto and off of the MSAS 402 and as MSPs 306 become otherwise unavailable (e.g., enter into secure medical conferences with consumers). Accordingly, the direct connection aspect of the system 300 enables the available provider list to accurately reflect the current availability of MSPs without needing to route messages and status updates through the MSAS server 402. In other embodiments, the consumer 304 may consult a web page or other source to locate an available MSP. Such a web page or other source may be made available by the MSAS 402 or another server.

In the present example, the QCG 302 and consumer 304 are buddies and the consumer's device directly notifies the QCG's device that the consumer is online and available for contact. As such, a communication session may be established between the consumer 304 and QCG 302 to discuss the child's condition in step 420. The session may be initiated by either the consumer 304 or the QCG 302.

In step 422, the consumer 304 may send a message directly to the MSP 306 requesting a medical consultation. If the MSP 306 grants the request, a communication session may be established directly between the devices of the consumer 304 and MSP 306. In the present example, the QCG 302 directly notifies the MSP 306 of the QCG's online status and establishes a communication session with the MSP. It is understood that each device may send messages directly to the other two devices involved in the medical consultation. In some embodiments, the devices may join the two sessions so that, for example, the device of the consumer 304 sends a single message (e.g., a broadcast) to both the QCG 302 and MSP 306 rather than sending two separate messages. If the QCG 302 is not a buddy of the MSP 306, a buddy request may be sent from the QCG to the MSP as described in previously incorporated U.S. patent application Ser. No. 11/081,068. Once the buddy request is accepted by the MSP 306, the QCG 302 may establish the session. In the present example, all registered entities of the medical conferencing services provider are buddies, although restrictions may be imposed on the ability of one user to contact another user as previously described.

In step 430, once the various communication channels are established, the consumer 304, QCG 302, and MSP 306 may discuss the child's condition, take and review measurements, and perform other actions that would typically require the physical presence of multiple attendees of the session. After the session has been completed, one or more of the consumer 304, QCG 302, and MSP 306 may terminate their involvement in the secure multi-party medical conference by ending the session or logging off. For example, the MSP 306 may log off the MSAS 402 or change its status to available to become available for another consultation. Such status changes may be sent directly to all buddies and to the MSAS 402. Logs or other recordings of the session may be saved in the database for later review, although such logs may need the permission of one or more of the session attendees before being recorded and/or saved.

Referring now to FIG. 4b, one embodiment of a method 450 for establishing secure multi-party medical conferencing is illustrated. To continue the previous example, the child of the consumer 304 attends a school having a QCG 302 (e.g., a nurse). The method 450 is illustrated as being divided by vertical dashed lines into three areas representing QCG actions, consumer actions, and medical conferencing services provider/MSP actions. It is understood that these three areas are illustrative only and the actions contained in each area may be moved to another area in other embodiments or the differentiation between areas may be removed entirely. The QCG 302 is registered with the medical conferencing services provider. The child suffers from a chronic illness. Artisans of skill will appreciate that the illustration of a chronic illness is exemplary and the child could be suffering from an typical illness or injury. In step 452, the child informs the QCG 302 that the child is not well.

In step 454, the QCG 302 determines whether or not parental authorization for medical care is required. For example, since this is a chronic illness, the QCG 302 may have previously been informed by the consumer 304 regarding the illness and a course of treatment for the child, or may be pre-authorized to take certain actions (e.g., may be authorized to contact an MSP 306 for treatment without contacting the consumer). Alternatively, the child may be above the age of minority in the state/country and so may be eligible to authorize medical care on their own behalf. For example, the school may be a college where, though the child is still covered by the consumer's insurance and the consumer's subscription with the medical conferencing services provider, the child is legally permitted to seek and approve their own medical care. Although not shown, if the child is covered under the consumer's subscription, the QCG 302 may send a request authorization message including an authorization code to the consumer 304 to verify that the coverage is valid or may verify its validity with the medical conferencing services provider itself.

If no authorization is required, the method 450 moves to step 456 and the QCG 302 accesses the medical conferencing services provider via the MSAS 402 and views the list of available MSPs. In step 458, the QCG 302 may select one of the MSPs from the list and send a request for consultation as described previously with respect to FIG. 4a. For example, the QCG 302 may request a secure medical conference with an available MSP 306 from the list by selecting a link corresponding to the available MSP 306 from the list of available MSPs or by otherwise selecting the desired MSP. In step 460, the MSP 306 accepts the request for the secure medical conference by, for example, selecting an “accept” link on the MSP terminal display. In some embodiments, the MSP 306 may deny the request for secure medical conference by selecting a deny link. In such embodiments, the QCG 302 may receive a message indicating that the MSP 306 is unavailable for a consultation. Upon acceptance, the method 450 moves to step 462, where the MSP 306 and QCG 302 enter a secure multi-party medical conference session using direct communications as described previously.

Returning to step 454, if the QCG 302 determines that parental authorization is required for medical treatment of the child, the QCG 302 contacts the consumer 304 in step 464. For example, the QCG 302 may contact the consumer 304 by telephone, pager, e-mail, or any other method of communication. The QCG 302 informs the consumer 304 of the illness/injury. In the present example, the method 450 then moves to step 466, where the consumer 304 signs onto the medical conferencing services provider via the MSAS 402. Following step 466, the method 450 may continue through steps 456, 458, 460, and 462 as previously described.

In some embodiments, the QCG 302 may sign onto the medical conferencing services provider via the MSAS 402 in step 468 and be selected by the consumer 304 in step 470 to join in the medical consultation. Alternatively, the QCG 302 may select the consumer 304 from a list or may join the medical consultation 462 in other ways (e.g., by request of the MSP 306).

During the medical consultation, the MSP 306 may require access to the child's medical history. Accordingly, in step 472, the consumer 304 may provide an access code to the MSP 306 or directly to the medical conferencing services provider (e.g., to the MSAS 402) to authorize the MSP 306 to access the child's medical records. In embodiments where the access code is provided to the MSAS 402, the MSP 306 may send a message to the MSAS 402 requesting access to the child's medical records. In response, the MSAS 402 may prompt the consumer 304 for the access code. The prompting of the consumer 304 to provide the access code to the MSAS 402 may serve both to inform the consumer 304 that access to the child's medical records is being sought by the MSP 306 and to provide the consumer 304 with the ability to verify that the MSP 306 is authorized to access the child's medical records.

The access code for the child's medical records may uniquely identify the medical records to prevent additional records from being disclosed. Furthermore, the child's medical records may be flagged or otherwise segmented to allow the consumer 304 to control the release of only portions of the medical records. In some embodiments, the consumer 304 may be provided with a separate access code for each dependent associated with the consumer's account with the medical conferencing services provider. After the consumer 304 provides the access code to the MSAS 402, the MSAS may send a message to the MSP 306 informing the MSP 306 that access to the child's medical records has been granted or the MSAS may simply send the medical records (or portions thereof) to the MSP. In some embodiments, the MSP 306 is provided a copy of a portion of the child's medical records electronically such that the MSP 306 can print the portion for closer review.

During and after the secure multi-party medical conference session, the MSP 306 may make relevant notes regarding the content of the consultation, diagnosis, if any, medications or treatments prescribed, and any courses of action conducted or recommended. The MSP 306 may then save a copy of the notes to the child's medical records stored on the database. For example, the MSP 306 may save the notes to the medical records by retaining a link for “saving” on the MSP terminal. Additionally, the MSP 306 may save the notes by providing a record identifier, such as the consumer's user ID or the access code. In this fashion, the MSP may retain a physical copy of the medical records as well as update the medical records stored on the database.

If no medical records are needed, the method 450 may move directly from step 462 to step 474. In step 474, a determination may be made as to whether the consultation is over. If not, the method 450 moves to step 476, where the consultation continues. Steps 474 and 476 may be repeated until the consultation ends in step 478. It is understood that the consultation may end for one party (e.g., the QCG 302) while continuing for the other parties (e.g., the consumer 304 and the MSP 306).

Referring to FIG. 5, one embodiment of a network 500 includes an MSAS (e.g., the MSAS 402 of FIG. 4) coupled to the packet network 308 (FIG. 3). The MSAS 402 may be used to provide secure medical conferencing services by a medical conferencing services provider. For purposes of illustration, a consumer terminal 502 is identical or similar to the device described previously with respect to the consumer 304 of FIG. 3. The consumer terminal 502 may be coupled to the medical conferencing services provider (e.g., to the MSAS 402) via the packet network 308. In the present example, the network portion supported by the medical conferencing services provider includes not only the MSAS 402, but also a medical records database 504 accessible via the packet network 308 and/or coupled to the MSAS 402 via another connection that does not pass through the packet network 308. It is understood that the medical records database 504 may be distributed, combined with the MSAS 402, or otherwise configured. Furthermore, the medical records database 504 may be under the control of a third party rather than the medical conferencing services provider.

A plurality of MSP terminals 506, each of which is identical or similar to the device described previously with respect to the MSP 306 of FIG. 3, may have the ability to couple to the MSAS 402, medical records database 504, consumer terminal 502, and other MSP terminals 506. Each connection may be wireless and/or wired, and the consumer terminal 502 and MSP terminals 506 may represent multiple terminals. For example, a single MSP terminal 506 may represent an office having multiple MSP terminals 506 located therein. It is understood that each consumer terminal 502 and MSP terminal 506 may perform log in procedures as previously described to gain access to services provided by the medical conferencing services provider via the MSAS 402.

The medical records database 504 may enable an MSP terminal 506 to gain access to medical information relating to a consumer (e.g., the consumer 304 of FIG. 3) if certain conditions, such as access permissions, are met. As described previously, when the consumer 304 subscribes to the medical conferencing services provider, the consumer provides information to complete a patient profile. Additionally, the consumer 304 or another party (e.g., the consumer's physician (MSP 306 of FIG. 3)) may provide a copy of the consumer's medical records to the medical conferencing services provider or a third party (e.g., a third party operating the medical records database 504). Alternatively, the consumer's medical records may already be in the medical records database 504 and the consumer 304 or the consumer's MSP 306 may simply give permission to make the records available.

The consumer's medical records may be provided electronically (e.g., via an electronic record or filling out an online form) to the MSAS 402 for storage, may be provided directly to the medical records database 504, or may be sent (electronically or on a printed medium) to the medical conferencing services provider. If sent electronically to the MSAS 402, the MSAS may store the medical records in the medical records database 504 in a manner that links the medical records to the profile of the consumer 304. If a physical copy of the medical records is provided, the medical conferencing services provider may process and scan the medical records securely and in accordance with applicable regulations (e.g., HIPAA requirements) and then store an electronic version of the medical records in the medical records database 504 in a manner that links the medical records to the profile of the consumer 304. In some embodiments, the medical conferencing services provider may store consumer profiles in a first database and consumer medical records in a second database. In other embodiments, the medical conferencing services provider may store consumer profiles and consumer medical records in a single database.

Referring to FIG. 6a, a block diagram 600 illustrates one embodiment of the MSAS 402 and medical records database 504 in greater detail. In the present example, the medical records database 504 stores a consumer access code 602, consumer medical records 604, and consumer profile 606. Although not shown, the MSAS 402, medical records database 504, or another storage entity stores profiles of the MSPs 506 and other profiles, such as profiles associated with QCGs as previously described. For purposes of illustration, the terms “consumer 304” and “consumer terminal 502” may be used interchangeably in the present example, as may the terms “MSP 306” and “MSP terminal 506.”

The access code 602 may be provided by the consumer 304 via the consumer terminal 502 or to the consumer by, for example, the MSAS 402 during initial registration or at a later time. The access code 602 may uniquely correspond to the consumer medical records 604. To ensure that the access code is unique, the MSAS 402 may compare the access code 602 against existing access codes stored in the medical records database 504 corresponding to existing medical records. If the access code 602 supplied by the consumer 304 matches an existing access code stored in the medical records database 504, the consumer 304 may be prompted to supply another access code 602. This process may repeated until the consumer 304 supplies a unique access code 602, i.e., a code that does not match an existing access code stored in the medical records database 504. Thereafter, the access code 602 supplied by the consumer 304 is stored in the medical records database 504 such that the access code 602 uniquely corresponds to the medical records 604 of the consumer 304.

Accordingly, during a medical consultation with the consumer 304, the MSP 306 may have access to the consumer's medical records 604. In some embodiments, the MSP 306 may be authorized to access the consumer medical records 604 only after the consumer 304 grants permission for that particular access request. For example, when the consumer terminal 502 and a first MSP terminal 506a may establish a secure medical conference, the consumer 304 may provide the access code 602 to the first MSP 306a. The access code 602 may authenticate the identity of the consumer 304 and provide the first MSP 306a access to the consumer medical records 604. The medical conferencing services provider and software on the MSP terminal 506 may be configured to provide a link to the consumer medical records 604 when the first MSP 306a and the consumer 304 establish the secure medical conference. The first MSP 306a may submit a request to access the consumer medical records 604 by selecting the link and then providing the access code 602. Additionally, if a second MSP 306b enters the conference, either the consumer 304 or the first MSP 306a can provide the access code 602 to the second MSP 306b. The second MSP 306b can then submit a request to access the consumer medical records 604 in the same manner as the first MSP 306a.

In other embodiments, the first MSP 306a may submit a request to access the consumer medical records 604 to the MSAS 402 and/or medical records database 504. In response to the request, the MSAS 402 and/or medical records database 504 may send a message to the consumer 304 notifying the consumer of the access request by the MSP 306a. The message may prompt the user 304 to provide the access code 602 to the MSAS 402 and/or medical records database 504 in order to give the MSP 306a access to the medical records 602. Accordingly, the message may both inform the consumer 304 that access to the consumer's medical records 604 is being sought by the first MSP 306a and provide the consumer 304 with the ability to validate that the first MSP 306a is authorized to access the medical records.

In still other embodiments, the consumer 304 may provide the MSP 306a with permission to access the consumer's medical records 604 prior to the conference. For example, the consumer 304 may enter access permission into the consumer profile 606, and the MSAS 402 and/or medical records database 504 may check the profile for permission. In such embodiments, if the profile does not contain access permission for the MSP 306a, the consumer 304 and/or the MSP 306a may then be prompted for permission.

In addition to accessing the medical records 604, the MSP 306 may modify the medical records to reflect a recent consultation. The modified medical records 604 may then be stored in the medical records database 502. Modifications to the medical records 604 may include comments indicating who performed the modifications. For example, the MSP's ID, date modified, and other information may be stored with the modified medical records 604 to provide a history of changes to the medical records.

It is understood that access to the consumer's medical records 604 may vary depending on system configurations of the medical conferencing services provider, regulations governing access to medical records, and similar issues. Accordingly, access to the medical records 604 may be controlled in different ways and may even vary for different consumers of the medical conferencing services provider. For example, regulations in one geographic area may vary from regulations in another geographic area, and the medical conferencing services provider may account for these variations in controlling access to the medical records 604 of consumers 304 in those areas. Furthermore, access to medical records 604 may be tailored by individual consumers based on the preference of those consumers and/or their MSPs. Accordingly, the medical conferencing services provider may configure access to medical records 604 in many different ways.

Even if the consumer 304 does not have access to the consumer medical records 604, the consumer 304 may still edit the consumer profile 606. For example, the consumer 304 may access a configuration module (not shown) on the MSAS 402 to change access rights and/or the access code 602 for the medical records 604. The consumer 304 may grant or revoke permission for the MSP 306a to access the medical records 604, configure settings on how the consumer is to be notified if someone requests access to the medical records, and perform similar access control functions.

Referring now to FIG. 6b, one embodiment of a device 620 that may be used by the QCG 302, consumer 304, and/or MSP 306 to connect to the medical conferencing services provider is illustrated. The device 620 may be a computer, personal digital assistant (PDA), cellular telephone, or any other device capable of transmitting, processing, and/or receiving signals via wireless and/or wireline communication links. The device 620 may include components such as a central processing unit (“CPU”) 622, a memory unit 624, an input/output (“I/O”) device 626, and a network interface 628. The network interface may be, for example, one or more network interface cards (NICs) that are each associated with a media access control (MAC) address. The components 622, 624, 626, and 628 are interconnected by one or more communication links 630 (e.g., a bus). It is understood that the device 620 may be differently configured and that each of the listed components may actually represent several different components. For example, the CPU 622 may actually represent a multi-processor or a distributed processing system; the memory unit 624 may include different levels of cache memory, main memory, hard disks, and remote storage locations; and the I/O device 626 may include monitors, keyboards, otoscopes, stethoscopes, and the like. The network interface 628 enables the device 620 to connect to a network, such as the packet network 308 of FIG. 3.

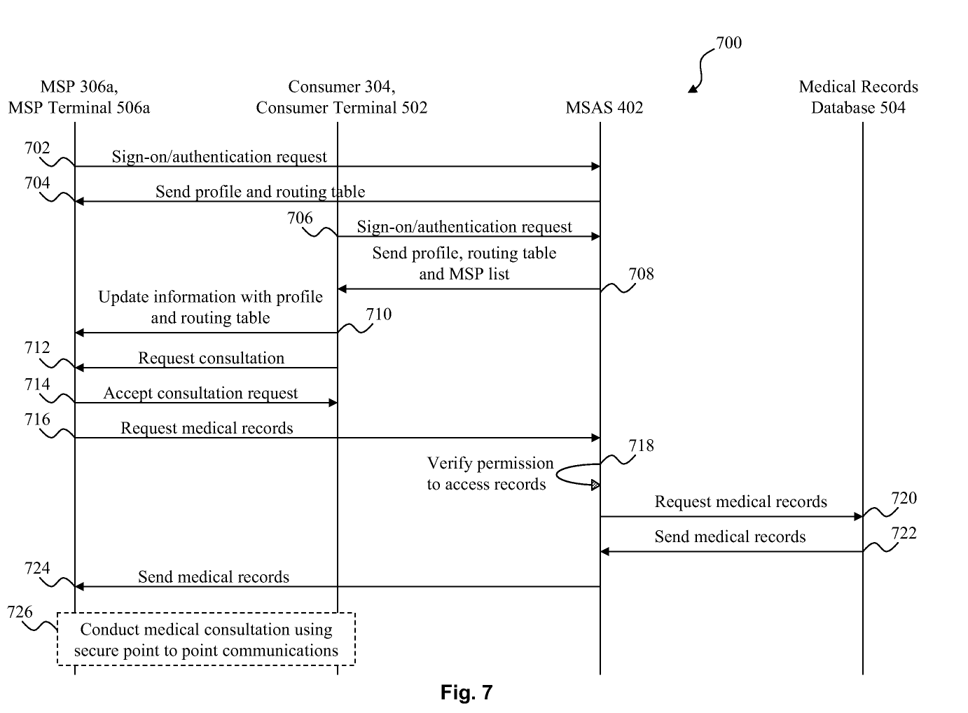

With additional reference to FIG. 7, a sequence diagram illustrates one embodiment of a method 700 by which a secure medical conference may be established between the consumer 304 and MSP 306a of FIG. 6a, and how the medical records 604 may be obtained by the MSP. In the present example, the consumer 304 and MSP 306a have registered with the medical conferencing services provider as described previously. Accordingly, when the method 700 begins, the consumer 304 and MSP 306a have terminals 502 and 506a, respectively, that are configured to execute software capable of connecting to the medical conferencing services provider, and each has established a profile and is authorized to provide and/or consume services offered via the medical conferencing services provider. One or both of the consumer 304 and MSP 306a may be an “endpoint” that is a “buddy” of other endpoints (including each other) in terms of previously incorporated U.S. patent application Ser. No. 11/081,068.

In step 702, the MSP 306a authenticates with the medical conferencing services provider by accessing the medical conferencing services provider via the packet network 308 (FIG. 3). The authentication process may include the MSP 306 providing the MSP ID and password to the MSAS 402. As described previously, the MSAS 402 updates the list of available MSPs with the information of the MSP 306 (not shown in FIG. 7). In step 704, the MSAS 402 responds to the MSP 306 with such information as profiles and one or more routing tables. In step 706, the consumer 304 authenticates with the medical conferencing services provider via the packet network 308 by, for example, sending a sign-on/authentication request to the MSAS 402 that includes the consumer's ID and password. In response and after verifying the consumer's authentication information, the MSAS 402 authenticates the consumer 304 as a valid member of the medical conferencing services provider and sends profile and routing table information to the consumer in step 708. In step 710, due to the point-to-point nature of the system in the present example, the consumer 304 may send a notification message directly to the MSP 306 without sending it through the MSAS 402.

In step 712, the consumer 304 requests a consultation with the MSP 306a after, for example, selecting the MSP from a list of available MSPs as previously described. In step 714, the MSP 306a may accept the request. If the MSP 306a denies the request, the method 700 may return to step 712 after the consumer 304 selects another MSP from the list of available MSPs.

In step 716, the MSP 306a requests the medical records 604 corresponding to the consumer 304 from the MSAS 402 and, in step 718, the MSAS verifies that the MSP 306a has permission to access the medical records. For example, the MSAS 402 may send a message to the consumer 304 as previously described, and/or may check the consumer profile 606 to determine if the MSP 306a has been given permission by the consumer. Once access is verified, the MSAS 402 requests the medical records 604 from the medical records database 504 in step 720, and the medical records database returns the medical records to the MSAS in step 722. In step 724, the MSAS 402 sends the medical records 604 to the MSP 306a. The consumer 304 and MSP 306a may then engage in the consultation session in step 726 with the MSP being able to refer to the consumer's medical records 604.

It is understood that the method 700 is only one example of how the medical records 604 may be accessed by the MSP 306a. Accordingly, many different variations are possible both with the system (e.g., the MSAS 402 and the medical records database 504 may be combined) and the process for verifying access permission.

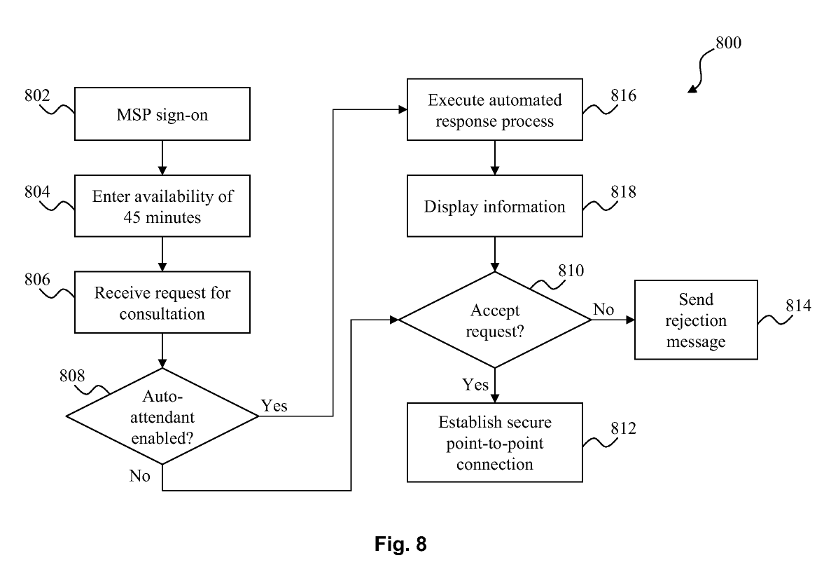

Referring to FIG. 8, one embodiment of a method 800 illustrates possible operations of an MSP 306 (e.g., the MSP 306 of FIG. 3). In the present example, it is understood that the MSP 306 has already registered with the medical conferencing services provider prior to the beginning of the method 800. In step 802, the MSP 306 signs on to the medical conferencing services provider and indicates a time window of availability for forty-five minutes in step 804. For example, an in-office patient may have been scheduled for a forty-five minute exam but then failed to appear for the exam. Therefore, the MSP 306 now has forty-five minutes of time available and no patient waiting in office. Accordingly, the MSP 306 signs on and indicates an availability of forty-five minutes. In some embodiments, the MSP 306 may choose to sign-on to the medical conferencing services provider for the entire day.

In step 806, the MSP 306 receives a request for a consultation via the medical conferencing services provider. In step 808, a determination is made as to whether the MSP 306 has enabled an auto-attendant module. For example, even though the MSP 306 may be examining in-office patients, the MSP 306 may enable the auto-attendant module to screen incoming requests for secure multi-party medical consultations via the medical conferencing services provider. The MSP profile may include editable information and settings for the auto-attendant module. For example, the MSP 306 may enable or disable the auto-attendant module, may record text, voice, and/or video for playback to a consumer, and otherwise configure the behavior of the auto-attendant within defined parameters. Accordingly, the MSP 306 may set the auto-attendant module to auto-answer mode (i.e., enabled) or do-not-answer mode (i.e., disabled).

If the auto-attendant is not enabled, the method 800 continues to step 810, where a determination is made as to whether to accept the consultation request. If the request is accepted, a secure point-to-point connection is established in step 812 between the MSP terminal and consumer terminal as previously described. If the request is rejected, a message indicating the rejection may be sent to the consumer in step 814.

If the auto-attendant is enabled as determined in step 808, the method 800 moves to step 816. In step 816, an automated response process (e.g., an interactive voice and/or video response process) may be executed. For example, when enabled for auto-answer mode, the auto-attendant module may provide an interactive voice and/or video response module that can query the consumer 304 for some basic information. The auto-attendant module may obtain the ID of the consumer 304 (e.g., from the request or by querying the consumer) and access the corresponding consumer profile to retrieve the consumer's name and demographic information. In some embodiments, the consumer 304 may configure the consumer profile 606 to provide only specified information (e.g., the consumer's name) to the auto-attendant module. If desired, the consumer 304 responds to the auto-attendant module with the scope and nature of the consultation sought. For example, the consumer 304 may state that the consumer is traveling and is experiencing flu-like symptoms. In step 818, the auto-attendant module may display the acquired information on the MSP 306 terminal, thereby informing the MSP 306 that, for example, consumer “John Smith is traveling and experiencing flu-like symptoms.” The method 800 then continues to step 810 and proceeds as previously described.

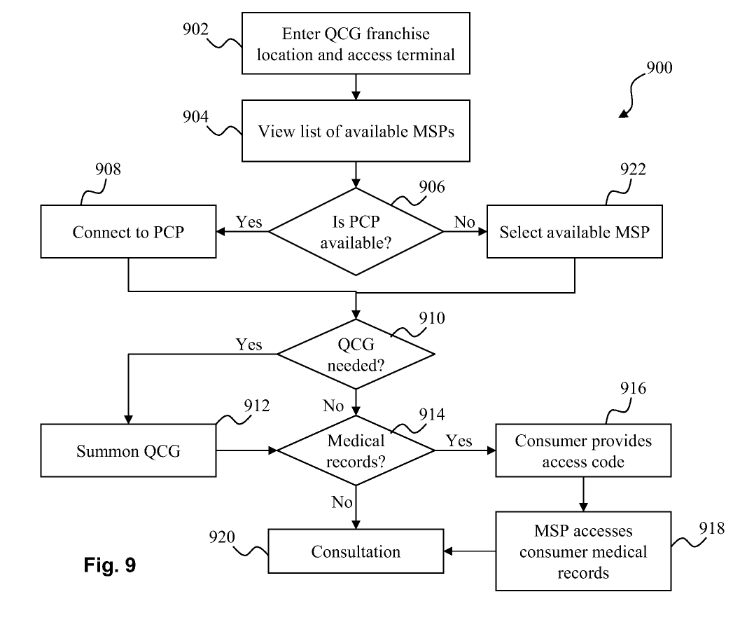

Referring to FIG. 9, in another embodiment, a method 900 illustrates that a consumer 304 may access the medical conferencing services provider via a consumer terminal provided by a QCG franchise. The QCG franchise may be a stand-alone office with a number of QCGs 302, such as nurses or medical technicians. The QCG franchise may be a stationary medical station, such as may exist in an office, hotel, business, retail location, or airport, or may be a portable medical station (e.g., in a vehicle) or a medical kiosk. The QCG franchise may have limited staffing and may be configured so that the consumer 304 can conduct the secure multi-party medical conference in private. The QCG franchise includes one or more consumer terminals and one or more QCG terminals configured to access the medical conferencing services provider. Although different software may be used on the consumer and QCG terminal (and on MSP terminals), it is understood that the software installed on each terminal may be identical and that differences in access type may be controlled by the medical conferencing services provider and tied to an individual consumer, QCG, and MSP ID.

In step 902, the consumer 304 enters a QCG franchise and requests access to a terminal. Such access may require the consumer 304 to sign in with the QCG franchise, speak with a QCG or staff member of the franchise, complete paperwork, or perform other tasks. If the consumer 304 is a subscriber to the medical conferencing services provider, the consumer 304 may be granted private access to a consumer terminal in the QCG franchise. The consumer 304 may then access the medical conferencing services provider using the consumer's ID and password as previously described. If not a subscriber, the consumer 304 may be required to become a subscriber or may be represented by a QCG 302 of the franchise. For example, a QCG 302 may access the medical conferencing services provider with the consumer 304 using subscriber information associated with the QCG franchise.

In step 904, the consumer 304 views a list of available MSPs. In the present example, the consumer 304 may have a primary care physician (PCP) and may review the preferred contact list to determine if the PCP is available. If the PCP is available as determined in step 906, the consumer 304 may then select the PCP and establish a secure point-to-point connection with the PCP via the medical conferencing services provider in step 908. In step 910, if the PCP determines that measurements or tests are needed (e.g., blood drawn or some other physical examination performed), a QCG 302 at the QCG franchise may be summoned in step 912 to assist the PCP in the medical conference.

In step 914, a determination may be made by the PCP as to whether the consumer's medical records 604 are needed. If so and the PCP does not have access as previously described, the consumer 304 may provide the access code 602 to the PCP in step 916 to grant permission to the PCP. The PCP may then access the medical records 604 in step 918. In some embodiments, the consumer 304 may provide the access code 602 directly to the MSAS 402 or to a QCG 302 of the QCG franchise. In step 920, the consumer 304 and QCG 302 (if needed) can communicate with the PCP via the medical conferencing services provider. The PCP may instruct the QCG 302 regarding the treatment of the consumer 304 or may instruct the consumer 304 to seek an MSP 306 for an in-office visit. The PCP may be able to modify the consumer's medical records 604 and save the modifications to the medical records database 504 (FIG. 5).

Returning to step 906, if the PCP is not available or if the consumer 304 does not have a PCP, the consumer 304 may select an available MSP 306 in step 922. The method 900 may then continue as described above.

In some embodiments, the consumer 304 may provide the consumer's user ID to a QCG franchise staff member upon entering the QCG franchise. The QCG staff member may verify that the consumer 304 is a valid subscriber of the medical conferencing services provider. The consumer 304 may be directed to a room where a QCG 302 performs initial screening that may include questions regarding symptoms and the taking of measurements such as temperature, blood pressure, and weight. Thereafter, the QCG 302 may access the medical conferencing services provider via a QCG terminal and select an MSP 306 for a secure medical conference. In some embodiments, the QCG 302 may search for and select a specific MSP 306, such as the consumer's PCP.

The QCG franchise may be a portable medical station, such as a vehicle with a mobile QCG terminal, operated by a QCG 302. The portable medical station may be configured so that it can travel to a consumer's location to treat the consumer 304. For example, the portable medical station may be contracted by a hotel, chain of hotels, home health care agencies, airport, business, or office to provide care as needed by customers, travelers, and employees. Furthermore, in some embodiments, the portable medical station may be located in the hotel, airport, business, or office. For example, the consumer 304 may be staying in a hotel and may contact the hotel's concierge due to an illness. The hotel concierge may contact the portable medical station to arrange a medical consultation for the consumer 304.

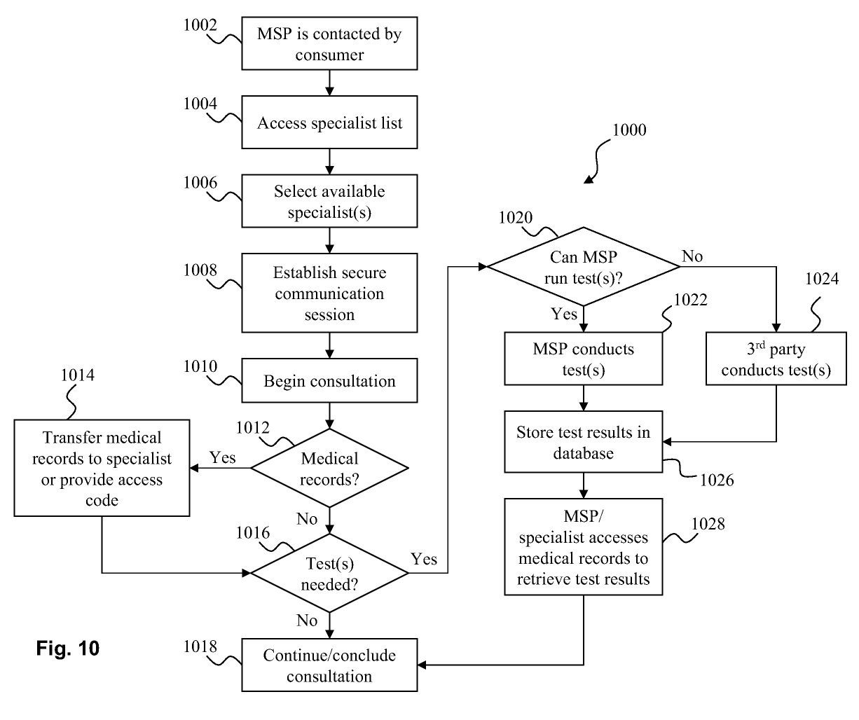

Referring to FIG. 10, in yet another embodiment, a method 1000 illustrates one way in which an MSP 306 may locate and contact a specialist using the medical conferencing services provider. In step 1002, the MSP 306 may be contacted (e.g., visited in person or via the medical conferencing services provider or another communication channel) by the consumer 304, who may or may not be subscribed to the medical conferencing services provider. The MSP 306 may perform an exam of the consumer 304 or have the exam performed by a QCG 302. In the present example, the MSP 306 determines that the opinion of a specialist needed. In a typical medical setting, the MSP 306 would often issue a referral to the consumer 304 by recommending a specialist or recommending that the consumer find their own specialist. The consumer 304 then would schedule an appointment with the specialist, which would often require that the consumer 304 take more time from work or other activities to attend the scheduled appointment. Such a specialist may then require additional tests to be performed on the consumer 304, which may require the consumer to go to yet another location that may also require an appointment. Accordingly, the consumer 304 may be required to use even more of their time to get the tests performed.

In contrast to the time-consuming and often frustrating process described above, in the present embodiment, the MSP 306 accesses a specialist provided by the medical conferencing services provider in step 1004. The specialist list may be similar to the MSP list or may be a version of the MSP list sorted by specialty. In some embodiments, the specialist list includes a duration for which a particular specialist will be available for consultations. For example, the specialist may have contracted with the medical conferencing services provider or another service to be available via the medical conferencing services provider at specified times. The specialist list may be updated at specified times or the list may be updated when a specialist signs on or off the medical conferencing services provider. As described with respect to the MSP list previously, the specialist list may be downloaded when the MSP 306 logs on to the MSAS 402. The MSP 306 may access the specialist list while the consumer is still in the session with the MSP.

After selecting an available specialist from the specialist list in step 1006, the MSP 306 may establish a secure point-to-point communication session with the specialist in step 1008. It is understood that establishing the channel may include sending a request directly to the specialist (e.g., without sending the request through the MSAS 402) and receiving a response directly from the specialist. Once this process is complete (assuming the specialist agrees to the request), the secure communication session is established between the MSP 306 and the specialist. As with previous embodiments, the communication session may include voice, video, data, or any other type of information and may be encrypted.

In step 1010, the medical consultation may occur between the MSP 306 and the specialist. In step 1012, a determination may be made as to whether the consumer's medical records (e.g., the medical records 604 of FIG. 6a) are needed by the specialist. In the present example, the MSP 306 already has access to the medical records 604, but it is understood that this may not be true in some embodiments. If the MSP 1012 does not have access to the medical records and such access is needed, the MSP may gain access as described with respect to FIG. 7. In step 1014, if the medical records 604 are needed by the specialist, the medical records may be transferred to the specialist (e.g., by the MSP 306) or the specialist may access the medical records via the MSAS 402. As described previously with respect to access by the MSP 306, if accessed via the MSAS 402, the access code may be provided to the specialist by the consumer 304 or MSP 306, and/or the consumer or MSP may be prompted to enter the access code when notified by the MSAS 402 of the access attempt.

When the specialist has been granted access, the specialist may have the ability to view all or a portion of the medical records 604. In some embodiments, to prevent the specialist from viewing all of the medical records 604, the consumer 304 or MSP 306 may flag or otherwise denote portions of the medical records that are to be viewable or not viewable by the specialist. In such embodiments, the consumer 304 or MSP 306 may create a temporary access code corresponding to the flagged portions of the medical records 604. In other embodiments, the MSP 306 may access and download some or all of the medical records 604 and send the downloaded information to the specialist via the secure communications session provided by the medical conferencing services provider.

In step 1016, after transfer of the medical records 604 or directly from step 1010 if no medical records are transferred, a determination may be made as to whether one or more tests should be performed on the consumer 304. For example, the specialist may instruct the MSP 306, QCG 302, or other medical staff or personnel at the location of the consumer 304 to take special measurements of the consumer or look for specific symptoms. As the consultation may occur in real time with the consumer 304 present with the MSP 306 or QCG 302, the consumer may be available for testing without requiring additional appointments. Furthermore, as the MSP 306 and specialist may both discuss the tests with the consumer 304 simultaneously, any issues may be addressed at once rather through later appointments. If no tests are needed, the consultation may continue and conclude in step 1018.

If it is determined in step 1016 that tests are needed, the method 1000 moves to step 1020, where a determination is made as to whether the MSP 306 or QCG 302 (e.g., medical staff or personnel at the location of the consumer 304) can conduct the tests. If it is determined that the MSP 306 can conduct the tests, the method 1000 moves to step 1022. In step 1022, the MSP or medical staff at the MSPs location conducts the tests. If it is determined that the MSP 306 can not conduct the tests, the method 1000 moves to step 1024, where a third party (e.g., a laboratory separate from the MSP 306) conducts the tests.

In step 1026, after the tests are conducted by either the MSP 306 or third party, the results may be stored in the medical records 604 of the consumer 304. The test results may be stored in the database 504 by the MSP 306/third party or may be sent (e.g., mailed or electronically transferred) by the MSP/third party to the medical conferencing services provider. For example, the third party may be registered with the medical conferencing services provider as a testing services provider (which may or may not be an MSP). Accordingly, the third party may be provided restricted access to the database 504 to store, but not access, information in the consumer's medical records 604. In such embodiments, the third party may store the test results in the medical records 604 using the consumer's ID. In other embodiments, the third party is not provided access to the consumer medical records and may send the test results to the MSP 306 or specialist, who may then store the test results. It is understood that the consumer 304 may need to go to the third party's location for the testing. Furthermore, the test results may take time to obtain, and so the current consultation may end and another consultation may be started at a later time to discuss the test results.

In step 1028, the stored test results may be accessed by the MSP 306 and/or specialist as previously described. The consultation may then continue and/or conclude in step 1018 with a discussion of the test results. At the conclusion of the consultation, the MSP 306 and/or specialist may modify the medical records 604 to reflect the results of the consultation, including any prescribed medications or other treatment recommendations.

In some embodiments, the medical conferencing services provider may enable the MSP 306, QCG 302, and other parties (e.g., a specialist or third party tester) to record billing information for the medical services provided to the consumer 304. In such embodiments, the MSP 306, QCG 302, or other party may submit a claim for services rendered to the insurance company of the consumer 304. If the consumer 304 is a not a subscriber to the medical conferencing services provider (e.g., the consumer is a walk-in at a QCG franchise), the consumer may be required to provide the insurance information prior to receiving services. If the consumer 304 is a member of the medical conferencing services provider, the consumer's insurance information can be stored in the profile 606. If the consumer's insurance company is a member of the medical conferencing services provider, it may then access the billing information stored in the database 504. The access provided to the insurance company may be limited to such billing information to prevent the insurance company from accessing the medical records of the consumer 304.

In some embodiments, a pharmacy, a franchise of pharmacies, or a store having a pharmacy therein may register with the medical conferencing services provider. During registration, the pharmacy may provide demographic information and a listing of medications that the pharmacy carries. Once registered, the pharmacy is able to remain signed-on to the medical conferencing services provider throughout the regular hours of operation of the pharmacy. In some embodiments, the consumer 304 is able to use a secure point-to-point communication session to query a pharmacist at the registered pharmacy via the medical conferencing services provider regarding the complications or requirements of a medication that has been prescribed to the consumer. The pharmacy may also receive electronic prescriptions from MSPs 306 via the medical conferencing services provider. In some embodiments, the consumer 304 may use the medical conferencing services provider to provide payment and insurance information to the pharmacy to facilitate the purchase of medications. In such embodiments, the pharmacy may arrange for delivery of the medications to consumer 304 or provide options for pick-up from the pharmacy.

It will be appreciated by those skilled in the art having the benefit of this disclosure that the systems and methods described herein for secure multi-party medical conferencing may be used to provide a secure connection for the transmission of voice, video, data, or other information relating to the medical treatment of a patient between two or more parties. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. For example, steps from various flow charts and sequence diagrams described above may be combined or further separated. Thus, it is intended that the following claims be interpreted to embrace all such further modifications, changes, rearrangements, substitutions, alternatives, design choices, and embodiments.

What is claimed is:

1. A system for providing medical conferencing for a plurality of parties by a medical conferencing services provider, the plurality of parties including a medical service provider (MSP) and a consumer of medical services, the system comprising:
a database including an electronic storage medium having a plurality of records stored thereon, wherein the plurality of records includes a first profile, a first identifier, and a first password corresponding to the MSP, a second profile, a second identifier, and a second password corresponding to the consumer, and an MSP list containing a plurality of MSP identifiers corresponding to a plurality of MSPs on the MSP list, wherein one of the plurality of MSP identifiers is the second identifier, and wherein the MSP list includes at least one MSP identifier not added to the MSP list by the consumer; and
a medical services authentication server (MSAS) coupled to the database and a packet network, the MSAS including an electronic storage medium having a plurality of instructions stored thereon for execution by the MSAS, wherein the plurality of instructions includes instructions for:
receiving the first password from a first device via the packet network and authenticating the first device as authorized to access the medical conferencing services provider based on the first password;
receiving the second password from a second device via the packet network and authenticating the second device as authorized to access the medical conferencing services provider based on the second password;
sending the MSP list and corresponding status information to the second device via the packet network after authenticating the second device, wherein the corresponding status information includes a status of the first device corresponding to the first identifier as available for contact by the second device; and
sending routing information to the second device via the packet network after authenticating the second device, wherein the routing information includes network address information needed by the second device to establish a secure, point-to-point communication session directly with the first device.

2. The system of claim 1 wherein the MSAS further includes instructions for:
receiving a request from the first device for medical records corresponding to the second identifier, wherein the medical records are stored in the database;
determining whether the first device is authorized to receive the medical records;
retrieving the medical records from the database only if the first device is authorized to receive the medical records; and
sending the medical records to the first device.

3. The system of claim 2 wherein the MSAS further includes instructions for:
sending a message to the second device indicating that the first device is attempting to access the medical records;
receiving a response from the second device with a medical records access code corresponding to the consumer contained therein; and
determining whether the first device is authorized to receive the medical records by verifying that the medical records access code is valid.

4. The system of claim 3 wherein the response from the second device with the medical records access code indicates that only a portion of the medical records are authorized to be sent to the first device.

5. The system of claim 2 wherein the MSAS further includes instructions for:
determining whether the first device is authorized to receive only a portion of the medical records;
determining whether the request from the first device for medical records includes the authorized portion; and
sending only the authorized portion of the medical records to the first device.

6. The system of claim 2 wherein the MSAS further includes instructions for:
receiving a request to modify the medical records from the first device;
determining whether the first device is authorized to modify the medical records;
modifying the medical records as requested; and
storing the modified records in the database.

7. The system of claim 1 wherein the MSAS further includes instructions for:
sending the first profile to the first device after authenticating the first device; and
sending the second profile to the second device after authenticating the second device.

8. The system of claim 7 wherein the MSAS further includes instructions for:
receiving information from the first and second devices to modify the first and second profiles, respectively; and
updating the first and second profiles in the database based on the received information.

9. The system of claim 1 wherein the first profile includes at least one medical specialty associated with the MSP, and wherein the MSP list is searchable based on the at least one medical specialty.

10. The system of claim 1 wherein the MSAS further includes instructions for filtering the MSP list prior to sending the MSP list to the second device, wherein the MSP list is filtered based on at least one parameter obtained from the second profile.

11. The system of claim 1 wherein the MSAS further includes instructions for:
receiving a status change message from the first device indicating that the first device is logging off of the MSAS; and
updating the MSP list to indicate that the first device is not available.

12. The system of claim 1 wherein the second device includes instructions for:
displaying the MSP list and corresponding status information to the consumer;
receiving input from the consumer, wherein the input indicates that the consumer has selected the second identifier from the MSP list;
sending a message using the routing information to the first device based on the received input, wherein the message requests the establishment of a secure communication session between the first and second devices, and wherein the message includes routing information needed for the first device to respond to the second device.

13. The system of claim 1 wherein the second device includes instructions for displaying the MSP list based on at least one filtering parameter provided to the second device by the consumer.

14. The system of claim 1 wherein the first device includes instructions for sending a status change message directly to the second device indicating that the first device is logging off of the MSAS.

15. The system of claim 1 wherein the first device includes instructions for an auto-attendant configured to receive a request from the second device for the establishment of a secure communication session between the first and second devices, and to initiate a series of predefined responses upon receiving the request.

16. The system of claim 1 wherein the database is part of the MSAS.

17. A device configured to participate in medical conferencing comprising:
a display;
a network interface;
a processor coupled to the display and network interface;
a memory unit coupled to the processor and configured to store a plurality of instructions thereon for execution by the processor, the instructions including instructions for:
sending an identifier and a password via a packet network to a medical services authentication server (MSAS), wherein the identifier and password identify that a user of the device is authorized to access services provided by a medical conferencing services provider;
receiving a medical service provider (MSP) list and routing information corresponding to each MSP on the MSP list from the MSAS, wherein the MSP list contains only MSPs that the user is permitted to directly contact and wherein the MSP list includes MSPs that were not added to the MSP list by the user;
receiving input from the user to establish a secure, point-to-point connection with a selected MSP from the MSP list;
identifying routing information corresponding to the selected MSP from the received routing information;
sending a request via the network interface to another device corresponding to the identified routing information to establish a secure, point-to-point connection; and
displaying a result of the request to the other device on the display.

18. The device of claim 17 further comprising instructions for:
receiving a message from the MSAS that an access request for medical records corresponding to the user has been received by the MSAS;
displaying the message to the user;
receiving input from the user in response to the message, wherein the input includes an access code; and
sending the access code to the MSAS.

19. The device of claim 17 further comprising instructions for filtering the MSP list based on at least one input parameter received from the user.

20. A method for providing secure electronic medical conferencing for multiple parties over a packet network, each party located in a different geographic area, a first party of the multiple parties providing a plurality of medical services and a second party of the multiple parties seeking at least one medical service, the method comprising:
providing the second party access to a secure multi-party medical conferencing service by:
providing a user ID and password for the second party, and
authenticating the second party by the user ID and password;
providing an editable user profile for the second party, wherein the user profile includes:
a user access code,
user visual preference settings,
primary care physician, and
user demographic information;
providing a prescreened list of doctors available for multi-party medical conferencing, wherein the first party is included in the prescreened list of doctors;
enabling the selection of the first party from the prescreened list of doctors;
aiding in the establishment of a secure connection between the first and second parties; and
allowing only the first party, and not others from the prescreened list of doctors, access to a medical record of the second party, wherein the medical record of the second party is stored on a database located in a geographically different area than the location of the first or second parties.
